(19) United States
(12) Patent Application Publication
(10) Pub. No.: US 2015/0142007 A1
(43) Pub. Date: May 21, 2015

(54) MALE CIRCUMCISCION DEVICE AND ASSOCIATED SYSTEMS AND METHODS

(71) Applicant: University of Washington through its Center for Commercialization, Seattle, WA (US)

(72) Inventors: John N. Krieger, Seattle, WA (US); Michael R. Bailey, Seattle, WA (US); James M. Hotaling, Chicago, IL (US); Brian Macconaghy, Kent, WA (US); Francis Olson, Seattle, WA (US)

(21) Appl. No.: 14/399,143

(22) PCT Filed: May 17, 2013
(86) PCT No.: PCT/US2013/041720

§ 371 Date: Nov. 5, 2014

Related U.S. Application Data
(60) Provisional application No. 61/649,015, filed on May 18, 2012.

(51) Int. Cl. A61B 17/326

(52) U.S. Cl. CPC A61B 17/326; A61B 2019/481
USPC 606/118

(57) ABSTRACT

The present technology relates generally to surgical devices for male circumcision and associated systems and methods. In some embodiments, a circumcision device configured in accordance with the technology comprises an outer member configured to be positioned along an exterior portion of a prepuce adjacent a glans of a penis of a human patient. The outer member may include an open-ended band moveable between an unlocked position and a plurality of locked positions. The band includes circumferentially overlapping first and second ends in each of the locked positions, and defines a closed space having a different fixed diameter in each of the locked positions. The device can also include a cylindrical inner member configured to be positioned within the band of the outer member and adjacent an inner portion of the prepuce.

### CROSS-REFERENCE TO RELATED APPLICATIONS

This application claims the benefit of U.S. Provisional Patent Application No. 61/649,015, filed May 18, 2012, entitled “SURGICAL GUIDE FOR AIDS PREVENTION,” which is incorporated herein by reference in its entirety.

### TECHNICAL FIELD

The present technology is generally related to surgical devices and methods of using surgical devices to remove tissue. In particular, several embodiments are directed to surgical devices for male circumcision and associated systems and methods.

### BACKGROUND

Male circumcision is defined as the surgical removal of all or part of the prepuce (foreskin) of the penis. Circumcision is practiced for various reasons, including as a medical procedure to treat or prevent infections, injury, or anomalies of the prepuce. Male circumcision, for example, is expected to result in statistically and clinically significant decreases in risk for a number of sexually transmitted infections (e.g., human papillomavirus, genital herpes, etc.) in circumcised men and their sexual partners (e.g., especially vaginitis and cervical infections). Circumcised neonatal males have a 90% decreased rate of urinary tract infections. Circumcised males have substantially decreased rates of penile cancer and have essentially no risk of complications associated with foreskin (i.e., phimosis, balanitis, and posthitis).

Since the 1980s, observational studies have also suggested an association between male circumcision and human immunodeficiency virus (“HIV”) infection in males. In 2005, the results of a South African randomized controlled trial of male circumcision for preventing HIV acquisition in heterosexual men were published, followed in early 2007 by the publication of Kenyan and Ugandan randomized controlled trial findings. These studies have effectively proven that male circumcision reduces human HIV transmission by approximately 65%.

Promoting male circumcision has become an important strategy to prevent and/or limit HIV infection risk among heterosexual men in developing countries, such as those comprising sub-Saharan Africa. Unfortunately, developing nations with high rates of HIV lack adequate numbers of trained practitioners to provide adult male circumcision services, as well as the surgical materials required to perform the procedure. As an alternative to traditional surgical procedures that require substantial training and experience, several compression-based circumcision devices have been developed to help achieve the goals of providing cosmetic circumcision that can be performed by non-physician medical providers. Examples of conventional compression devices include the Shang Ring, Prepex™ (Circ Medtech, British Virgin Islands) and the TaraKlamp. Such devices compress a proximal, circumferential portion of the prepuce to induce necrosis of the preputial tissue. One drawback with such approaches, however, is that “slow compression” devices require patients to wear a plastic device continuously for at least one week to allow the preputial tissue to become necrotic prior to device removal. Furthermore, clinics or treatment facilities using slow compression devices require a substantial inventory of device sizes to accommodate various patients. Another drawback with this approach is that slow compression devices require a second visit to a physician for device removal, and recovery often takes longer than healing from a surgical wound. Studies have demonstrated that the lengthy treatment time and requirement for multiple visits to the physician associated with slow compression devices has negatively impacted the adoption rate for adult male circumcision.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure. Furthermore, components can be shown as transparent in certain views for clarity of illustration only and not to indicate that the illustrated component is necessarily transparent.

FIG. 1 is a schematic front view of a penis.

FIG. 2 is a schematic partial cross-sectional front view of a penis.

FIG. 3A is a side perspective view of a circumcision assembly configured in accordance with an embodiment of the present technology.

FIG. 3B is a side perspective view of the circumcision assembly of FIG. 3A as assembled in accordance with an embodiment of the present technology.

FIG. 4A is a side perspective view of an outer member configured in accordance with an embodiment of the present technology.

FIG. 4B is a top view of the outer member shown in FIG. 4A in the “open-unlocked” position.

FIG. 4C is a top view of the outer member shown in FIG. 4A in the “closed-unlocked” position.

FIG. 4D is a top view of the outer member shown in FIG. 4A in a “locked” position.

FIG. 5A is a front perspective view of an inner member configured in accordance with an embodiment of the present technology.

FIG. 5B is a front view of the inner member shown in FIG. 5A.

FIG. 6 is a circumcision assembly having a sizing guide configured in accordance with another embodiment of the present technology.

FIG. 7 is a side view of a handle configured in accordance with an embodiment of the present technology.

FIG. 8 is a perspective view of a circumcision assembly including a connector configured in accordance with another embodiment of the present technology

FIG. 9 is a perspective view of a circumcision assembly including a connector configured in accordance with another embodiment of the present technology

FIG. 10A is a schematic front view of an uncircumcised, non-erect penis showing a marked incision line.

FIG. 10B is a schematic front view of the outer member loosely positioned at the base of an uncircumcised, non-erect penis.

FIG. 10C is a schematic front view of the outer member positioned adjacent the glans and the inner member being placed through an opening in the prepuce to an interior portion of the prepuce.

FIG. 10D is a schematic front view of the circumcision assembly positioned on the penis before the prepuce is excised.

FIG. 10E is a top view of FIG. 10D.

FIG. 11 is a schematic view of a kit including a circumcision assembly, a cutting device, instructions for use, and packaging.

### DETAILED DESCRIPTION

The present technology is generally directed to devices, systems, and methods for male circumcision (e.g., partially or completely removing a prepuce of a penis of a human patient). In one embodiment, for example, a circumcision assembly includes an outer member configured to be positioned along an exterior portion of a prepuce adjacent a glans of the penis, and an inner member configured to be positioned within the outer member and adjacent an inner portion of the prepuce to compress preputial tissue therebetween. The prepuce can be cut circumferentially along a distal portion of the aligned outer and inner members and subsequently removed. During excision of the prepuce, the outer member can protect the penile shaft while the inner member can protect the glans of the penis. In some embodiments, the outer member can include an open-ended band that can be locked in one or more closed positions.

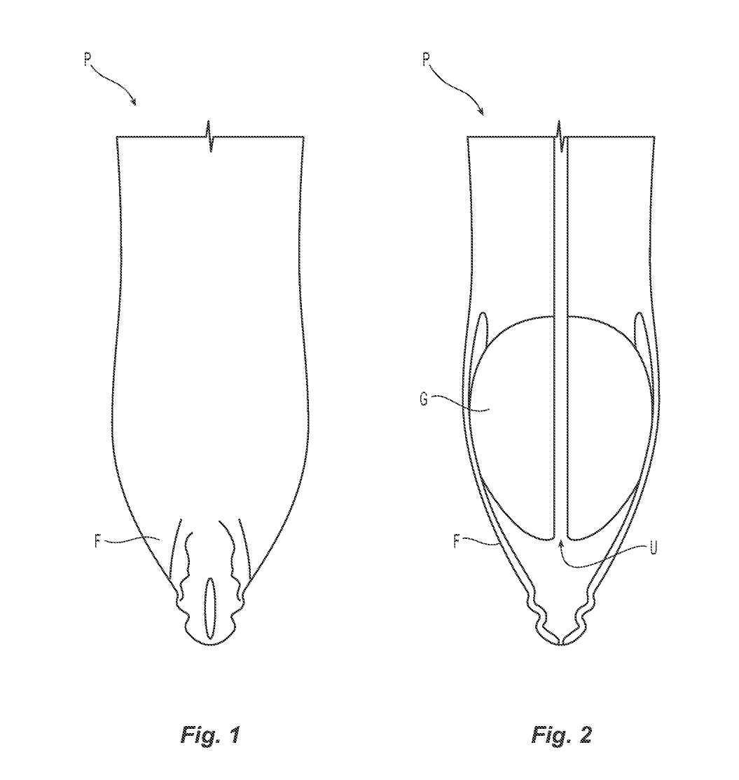

Specific details of several embodiments of the present technology are described herein with reference to FIGS. 1-11. Although many of the embodiments are described below with respect to devices, systems, and methods male circumcision, other tissue removal applications are within the scope of the present technology. Additionally, other embodiments of the present technology can have different configurations, components, or procedures than those described herein. For example, other embodiments can include additional elements and features beyond those described herein, or other embodiments may not include several of the elements and features shown and described herein.

For ease of reference, throughout this disclosure identical reference numbers are used to identify similar or analogous components or features, but the use of the same reference number does not imply that the parts should be construed to be identical. Indeed, in many examples described herein, the identically-numbered parts are distinct in structure and/or function.

### I. RELEVANT ANATOMY

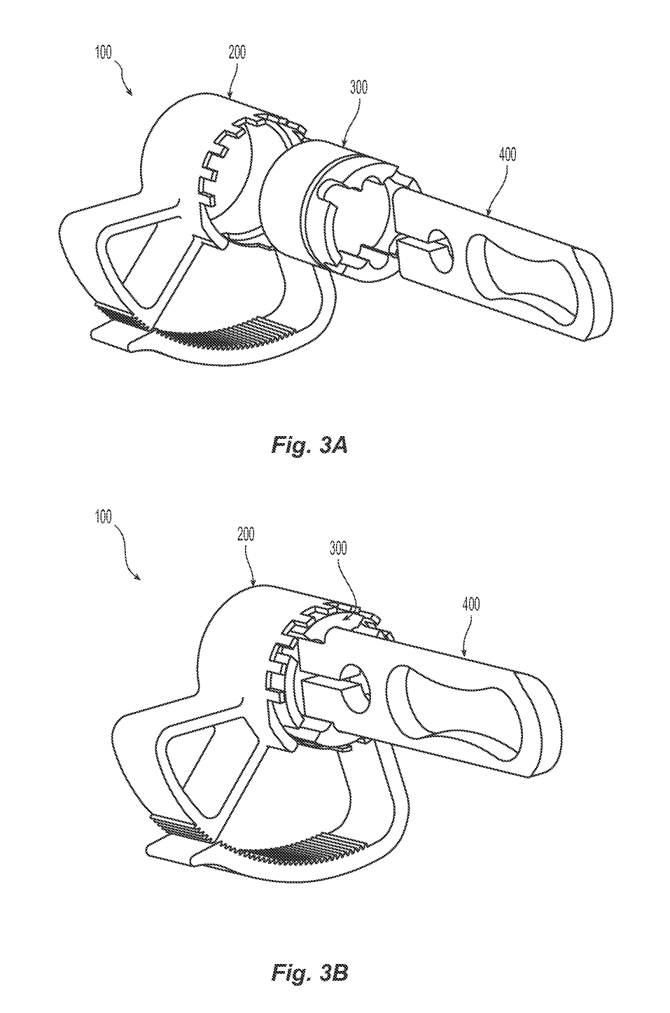

FIG. 1 is a schematic front view of a penis P, and FIG. 2 is a schematic partial cross-sectional front view of the penis P. Referring to FIGS. 1 and 2 together, the prepuce or foreskin F is a double-layered fold of skin and mucous membrane that covers the glans G of the penis P and protects the urinary meatus U when the penis P is not erect.

Generally, unless the context indicates otherwise, the terms “distal” and “proximal” within this disclosure reference a position relative to the patient's body along the shaft of the penis P. For example, “proximal” can refer to a position along the penile shaft closer to the base of the penis P, and “distal” can refer to a position that is more distant from the base of the penis P.

### II. SELECTED EMBODIMENTS OF CIRCUMCISION ASSEMBLIES

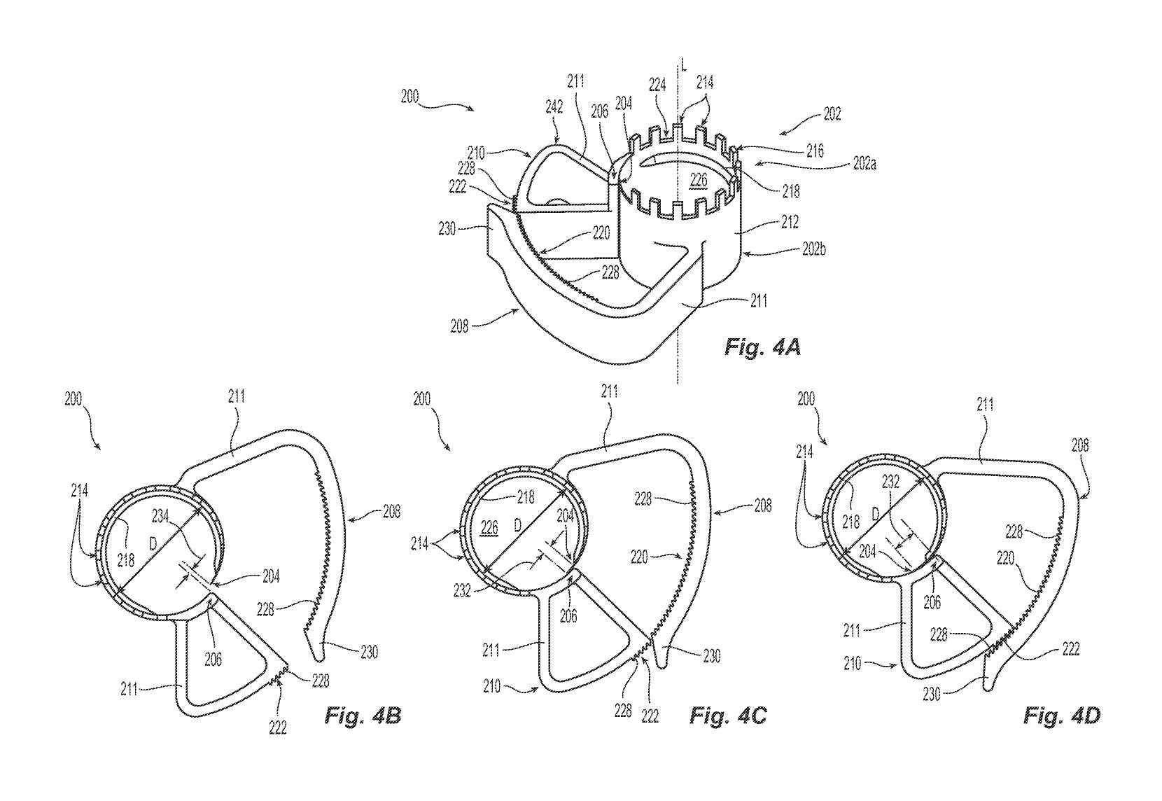

FIG. 3A is a side perspective view of a circumcision assembly 100 (“assembly 100”) configured in accordance with an embodiment of the present technology in a partially disassembled state and before application to a penis (not shown) of a human patient. FIG. 3B is a side perspective view of the assembly 100 in an assembled state before application. Referring to FIGS. 3A and 3B together, the assembly 100 includes a first or outer member 200, a second or inner member 300, and a handle 400. In some embodiments, the assembly 100 may not include the handle 400. As best seen in FIG. 3B, the inner member 300 can be positioned at least partially within the outer member 200, and the handle 400 can be detachably coupled to a distal portion of the inner member 300. Each component 200, 300 and 400 of the circumcision assembly 100 is described in greater detail below.

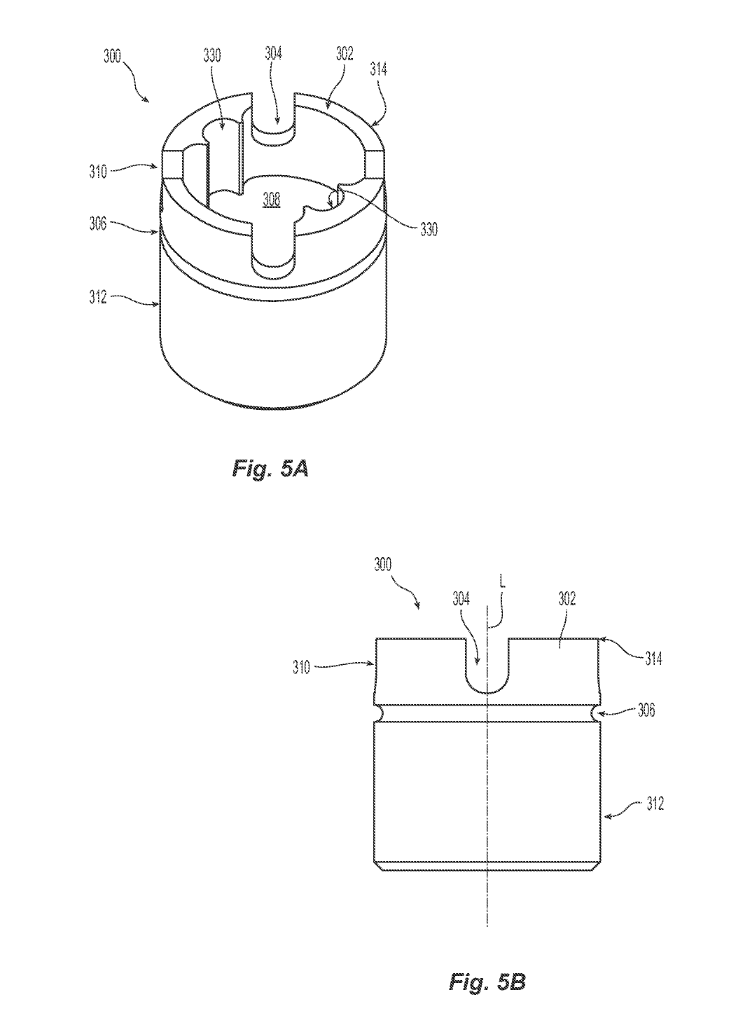

FIG. 4A is a side perspective view of the outer member 200 configured in accordance with an embodiment of the present technology, and FIGS. 4B-4D are top views of the outer member 200 in various states. Referring to FIGS. 4A-4D together, the outer member 200 can include an open-ended band 202 configured to be adjustably positioned around the shaft of a penis of a human patient (not shown) and/or an exterior portion of a prepuce adjacent a glans of the penis. The band 202 can include a generally cylindrical main body 212 having an adjustable diameter D, a first end 204, and a second end 206. The main body 212 is configured to protect the penile shaft and/or prepuce during the circumcision procedure.

The outer member 200 can further include a first arm 208 extending radially outwardly from the band 202 at or near the first end 204, and a second arm 210 extending radially outwardly from the band 202 at or near the second end 206. The radially extending arms 208, 210 can be quickly and easily engaged and controlled by a single hand of the clinician. For example, the first and/or second arms 208, 210 can have extensions 211 that extend generally perpendicular to the circumference of the band 202. Such perpendicular extensions 211 provide an accessible structure that the clinician's fingers can engage to leverage the first and second ends 204, 206 to affect the diameter D of the band 202.

The first and second arms 208, 210 can have a first and second fastening portion 220, 222, respectively, that individually include a plurality of engaging elements 228. When the first and second fastening portions 220, 222 are adjacent one another, the engaging elements 228 of the first arm 208 can engage the engaging elements 228 of the second arm 210 (or vice versa) and temporarily lock the arms 208, 210 relative to each other. In some embodiments, the engaging elements 228 can be ratcheting teeth, Velcro®, adhesive, magnets, and/or other suitable engaging elements and/or devices.

The band 202 is moveable between an “open-unlocked” position (FIG. 4B), a “closed-unlocked” position (FIGS. 4A and 4C), and a plurality of “locked” positions (FIG. 4D) by circumferentially opposing motion of the first and second arms 208, 210. For example, the first arm 208 may be moved clockwise while the second arm 210 moves counterclockwise, or the second arm 210 can be moved clockwise while the first arm 208 moves counterclockwise. As shown in FIG. 4B, when the band 202 is in the open-unlocked position, there is a gap 234 between the ends 204, 206 (and thus no circumferential overlap). When the first and second arms 208, 210 are moved in circumferentially opposing directions from the open-unlocked position, the first and second ends 204, 206 eventually come together such that the main body 212 of the band forms a closed, generally circular interior portion 226. In such a closed-unlocked position, the ends 204, 206 can either be touching without overlapping (not shown) or can form an overlapping portion 232 while the first and second fastening portions 220, 222 are not in contact and the arms 208, 210 are not locked (FIG. 4C). As the first and second arms 208, 210 continue to move in circumferentially opposing directions past the closed-unlocked position, the overlapping portion 234 between the two ends 204, 206 increases, the diameter (D) of the band 202 decreases, and the fastening portions 220, 222 of the first and second arms 208, 210 engage one another and lock the arms 208, 210 in place. Such overlapping ends 204, 206 ensure that the exterior portion of the shaft and/or prepuce of the patient's penis (not shown) are not pinched between the ends 204, 206 of the band 202 during adjustment and/or locking of the band 202.

To release the first and second arms 208, 210 and unlock the band 202, a lip 230 protruding from the first arm 208 can be pushed and/or pulled radially outwardly to release the engaged engaging elements 228. A clinician may desire to release the arms 208, 210, for example, to increase or decrease the band diameter D and re-lock (e.g., to tighten or loosen the band 202) and/or to remove the outer member 200 from the penile shaft (e.g., remove in a direction perpendicular to the shaft, remove longitudinally over the distal portion of the penis, etc.).

The band 202 can be adjusted to accommodate a wide range of shaft diameters. As noted previously, many conventional circumcision devices are not adjustable and clinics have to keep a relatively large inventory of various sized devices on hand to accommodate various patients. This approach is prohibitively expensive and cumbersome, particularly in many less-developed regions where promoting male circumcision has become an important strategy to prevent and/or limit HIV infection risk (e.g., sub-Saharan Africa). In contrast with conventional devices, however, the outer member 200 of the assembly 100 as disclosed herein can have one size that generally fits an entire adult population. It will also be appreciated that the assembly 100 may be used on infants, children, and/or adolescent patients. Accordingly, one configuration of the assembly 100 may be sized to generally fit an entire pediatric population, while another configuration may be sized to generally fit an entire adolescent population.

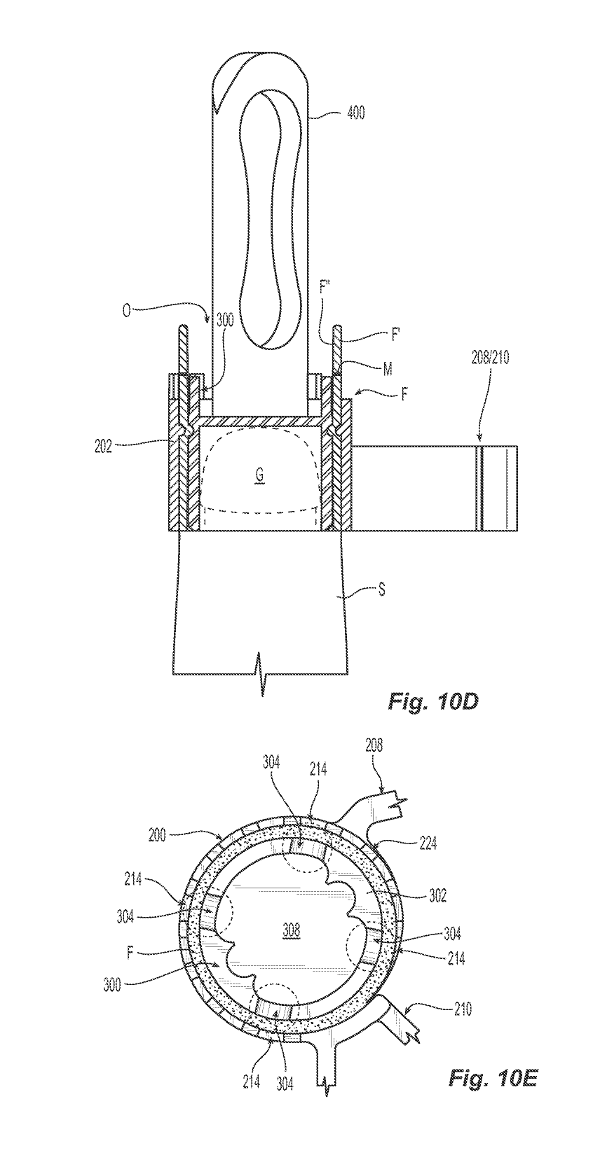

The band 202 may further include a plurality of castellations 214 extending longitudinally from a distal portion 202a of the main body 212. The plurality of castellations 214 may define a plurality of gaps 224 therebetween. In some embodiments, a distal edge 216 of the castellations 214 can define the distal edge of the band 202 and/or outer member 200. As discussed in further detail below with respect to FIG. 10A-10E, during the procedure, the distal edge 216 of the castellations 214 can be positioned at or just proximal to the planned incision line. Once the prepuce has been cut along the distal edge 216, the gaps 224 between the castellations 214 can be aligned with one or more gaps 304 of the inner member 300 and serve as suture guides to facilitate reattachment of the cut preputial tissue. The castellations 214 and/or gaps 224 on the band 202 can be arranged and/or configured to align with inner members 300 of different diameters.

In some embodiments, the band 202 can have a flange 218 extending circumferentially along at least a portion of the inner surface of the band's main body 212. The flange 218 can be configured to engage a groove 306 extending along an outer surface of the inner member 300, as discussed in greater detail below. The flange 218 may be located anywhere along a longitudinal axis L of the band 202.

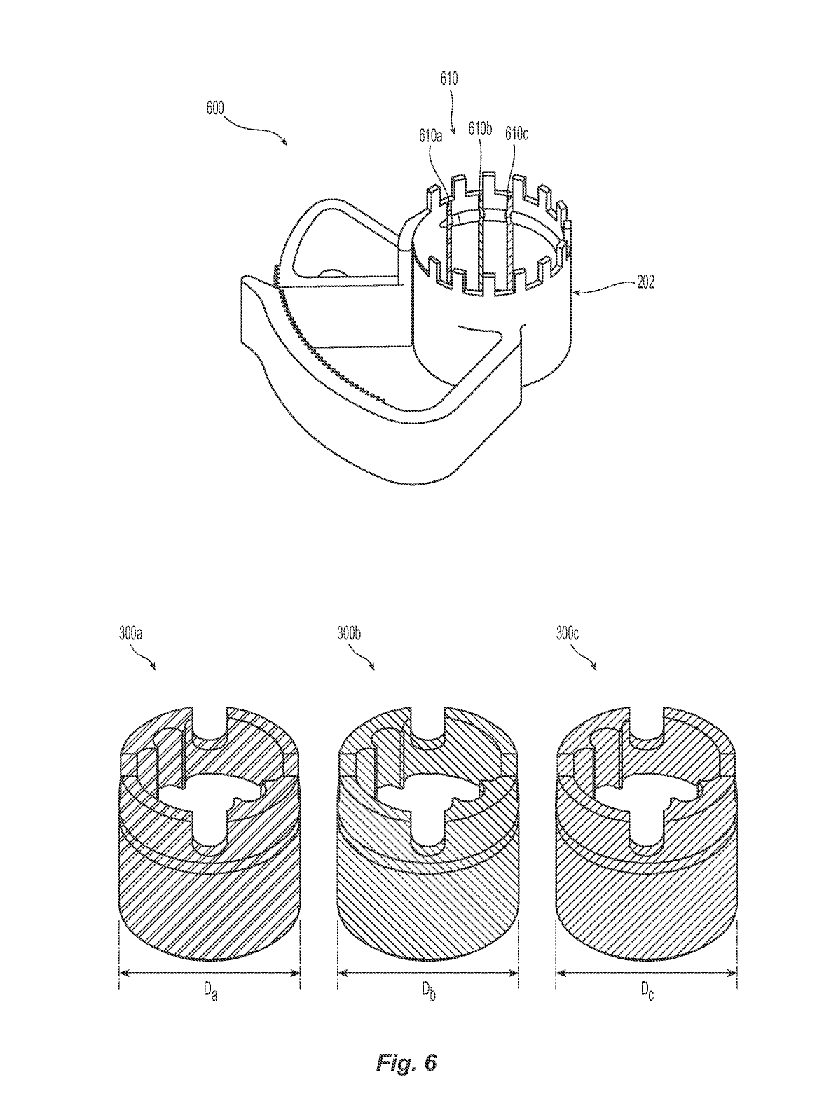

FIG. 5A is a front perspective view of the inner member 300 configured in accordance with an embodiment of the present technology, and FIG. 5B is a side view of the inner member 300. Referring to FIGS. 5A and 5B together, the inner member 300 is configured to be positioned at an inner portion of the prepuce around the glans (not shown). When the inner member 300 is positioned at a location adjacent the band 202 (FIG. 4A) positioned at an exterior portion of the prepuce, the band 202 can be tightened around the inner member 300 with the preputial tissue therebetween. Such continuous compression of the preputial tissue during the procedure not only stabilizes the assembly 100 with respect to the planned incision line, but also provides for improved hemostasis (e.g., blood control) during the circumcision, including eliminating the need for cautery or ties to achieve hemostasis (and the accompanying instruments). Many conventional surgical devices provide little, if any, compression and/or hemostasis of the preputial tissue during the procedure.

As mentioned above, in certain embodiments the inner member 300 can include a groove 306 configured to engage the flange 218 of the band 202 through the preputial tissue. The groove 306 may be located anywhere along the longitudinal axis L of the inner member 300. Such interaction between the band 202 and inner member 300 secures the inner member 300 relative to the band 202 and prevents slipping of the assembly 100 along the longitudinal axis L.

The inner member 300 can be generally cylindrical and include a distal region 310, a proximal region 312, and a cap 308 therebetween. Similar to the band 202 of the outer member 200 (FIG. 4A), the inner member 300 can have a plurality of castellations 302 extending longitudinally from the distal region 310 and defining a plurality of gaps 304 therebetween. In some embodiments, a distal edge 314 of the castellations 302 can define the distal edge of the inner member 300. The inner member 300 can have more castellations 302 and/or gaps 304 or fewer castellations 302 and/or gaps 304 than the outer member 200. In one particular embodiment, for example, the inner member 300 may have four evenly spaced castellations 302 that define four evenly spaced gaps 304, and the band 202 may have twelve castellations 214 and twelve corresponding gaps 224. In other embodiments, however, the inner member 300 may have a different arrangement and/or different features.

During a circumcision procedure, the inner member 300 can be positioned such that the distal edge 314 of the inner member 300 is generally aligned with the distal edge 216 of the outer member 200 to form a cutting guide. The cutting guide assures a high probability that excision of the prepuce will follow the planned and marked incision line and that excision will be perpendicular to the skin surface to facilitate healing. Additionally, one or more gaps 224 of the outer member 200 can be aligned with one or more gaps 304 of the inner member 300 to form one or more fastening guides that facilitate reattachment of the cut preputial ends. Further details are provided below with reference to FIGS. 10A-10E.

The cap 308 of the inner member 300 may comprise a solid disc arranged generally perpendicular to the longitudinal axis L of the inner member 300 between the sidewalls of the inner member 300. During a circumcision procedure, the cap 308 can be positioned at or distal to the distal portion of the glans (not shown) and protect the glans from the cutting device during the procedure. In some embodiments, the cap 308 may have a generally planar shape (see FIG. 5A), but in other embodiments the cap 308 can have generally convex shape to accommodate the curvature of the distal portion of the glans. Further, in some embodiments the inner member 300 may not include a cap 308 and instead define a continuous cylindrical lumen therethrough.

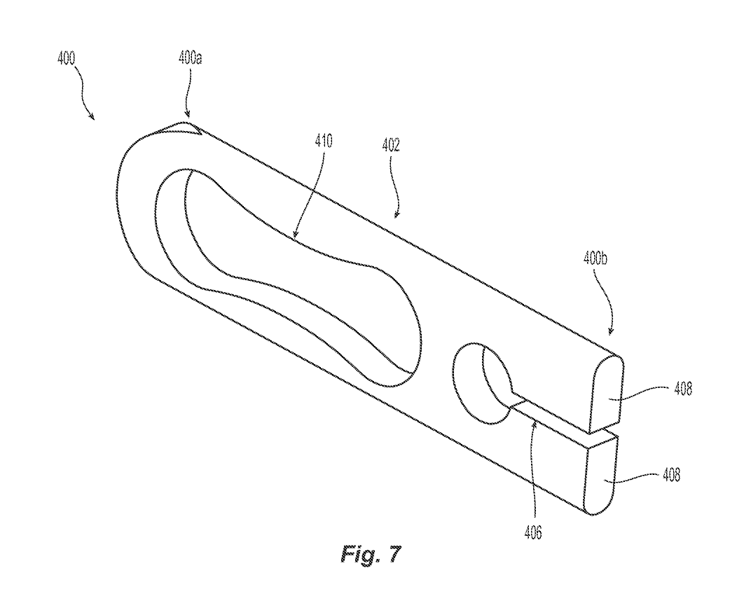

More often than not, one size inner member 300 can be used on a wide range of patients. In some embodiments, however, the circumcision assembly 100 can include a plurality of inner members, each having a different diameter. Likewise, the diameter D of the outer member 200 can be adjusted by the clinician to accommodate whichever size inner member 300 is being used. In certain embodiments, the outer member 200 can include a sizing guide to ensure the outer member 200 is tightened to sufficiently small diameter based on the size of the inner member 300 being used. For example, FIG. 6 is a partially-schematic view of an outer member 600 including a sizing guide 610 along an inner surface of the band 202. The sizing guide 280 can have a first zone 610a corresponding to an inner member 300a with a first diameter Dₐ, a second zone 610b corresponding to an inner member 300b with a second diameter D_{b} less than the first diameter Dₐ, and a third zone 610c corresponding to a third inner member 300c with a third diameter D_{c} less than the second diameter D_{b}. Individual zones and/or corresponding inner members can be demarcated by color, pattern, texture, symbol, letters, numbers, iterative notches, tactile feedback, and/or other communicative devices. In some embodiments, the sizing guide 610 can have more zones (e.g., four zones, five zones, six zones, etc.) or fewer zones (e.g., two zones), can be located on or at any portion of the outer member 200, and may have any suitable shape, length or general configuration. When the clinician is tightening the band 202 around the inner member with the prepuce therebetween, the clinician may align the first and/or second end 204, 206 of the band 202 with the zone corresponding to the matching inner member.

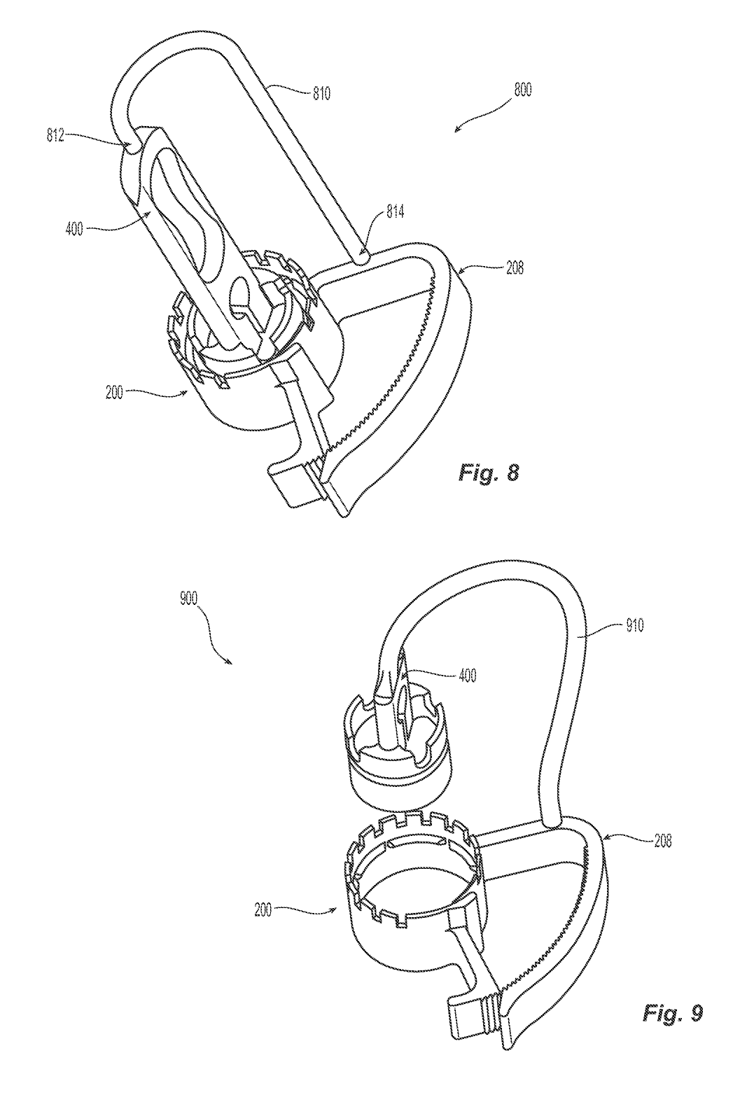

In certain embodiments, the inner member 300 may be configured to be coupled with the handle 400. FIG. 7, for example, is a side perspective view of the handle 400 configured to be detachably coupled to the distal region 310 of the inner member 300 (FIG. 5A). The handle 400 can have an elongated shape including a distal portion 400a, a proximal portion 400b, and a grip portion 402 therebetween. The proximal portion 400b may be bifurcated by a gap 406 such that the proximal portion 400b has two legs 408. Because of the gap 406, the legs 408 can flex towards one another to accommodate a range of inner member sizes. The elongated handle 400 is configured to extend the control and manipulation of the inner member 300 to a position adjacent and/or distal to the distal opening of the prepuce. The grip portion 402 of the handle 400, for example, can include a curvilinear slot 410 positioned to be gripped by a clinician during the procedure. In other embodiments, the slot 410 can have any suitable shape and/or configuration.

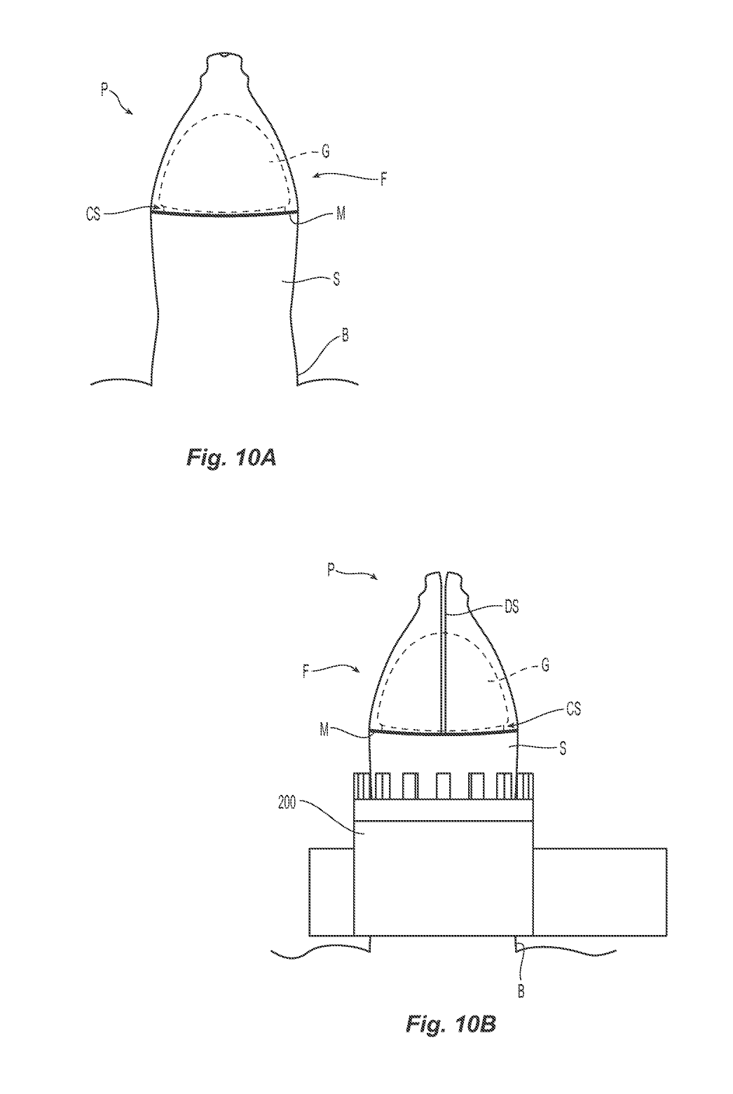

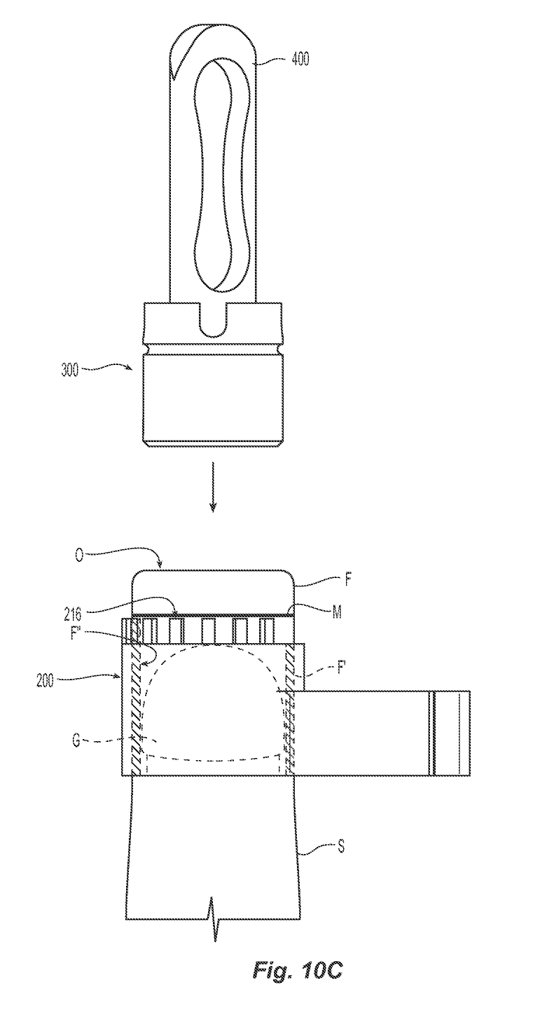

Referring to FIGS. 5A and 6 together, the distal region 310 of the inner member 300 includes opposing grooves 330 shaped and/or configured to receive the proximal portion 400b of the handle 400. The handle 400 can be removably coupled to the inner member 300 before and/or during the procedure. In some embodiments, however, the handle 400 may not be coupled to the inner member 300 at all. Additionally, as discussed in greater detail below with reference to FIGS. 8 and 9, the inner member may include an integrally formed or permanently coupled handle.

FIG. 8 is a perspective view of a circumcision assembly 800 configured in accordance with another embodiment of the present technology. As shown in FIG. 8, the circumcision assembly 800 can include a connector 810 having a first end 812 coupled to a distal region of the handle 400 and a second end 814 coupled to the outer member 200, thereby mechanically coupling the outer member 200 and the handle 400. In the illustrated embodiment shown in FIG. 8, the connector 810 is coupled to the arm 208 of the outer member 200, but in other embodiments the connector 810 can be coupled to any portion of the outer member 200. The handle 400 may be permanently attached to the inner member 300 and permanently attached to the connector 810, removably attached to the inner member 300 and permanently attached to the connector 810, or removably attached to the inner member 300 and removably attached to the connector 810. As shown in another embodiment of a circumcision assembly 900 in FIG. 9, the circumcision assembly 900 can have a shorter handle 400 and a longer, flexible connector 910 to accommodate the shorter handle 400.

In some embodiments the connector can be substantially rigid, and in other embodiments the connector can be flexible and/or elastic. The connector can be made from plastic, elastic, and/or other suitable materials.

### III. SELECTED METHODS FOR PERFORMING CIRCUMCISION

FIGS. 10A-10E illustrate a method for performing a circumcision using the circumcision assembly 100 in accordance with an embodiment of the present technology. Before and/or during use of the circumcision assembly 100, a clinician may administer antiseptic and local anesthesia at or near the penis. In some cases, general anesthesia may be used. Although standard anesthetic methods may be employed, the clinician may choose to administer premedication, analgesics, and/or topical agents to reduce the patient's pain during the procedure.

Referring first to FIG. 10A, before positioning the outer member 200, the clinician may optionally mark the planned incision by drawing a line M around the circumference of the exterior surface of the prepuce F and generally parallel to the coronal sulcus CS just proximal to the glans G. The outer member 200 is then placed around the penile shaft S and loosely positioned at the base B of the penis P, as shown in FIG. 10B. The clinician may optionally cut a dorsal slit DS (e.g., a single incision along the distal length of the prepuce F) to facilitate placement of the inner member 300. As shown in FIG. 10C, a clinician may then grasp a distal portion the prepuce F (e.g., via one or more tissue grasping devices such as a hemostat) and stretch the prepuce distally such that the marked incision line M originally sketched just proximal to the glans G stretches to a point at or near a distal portion of the glans G.

At this point, the outer member 200 can be shifted distally along the shaft S so that the outer member 200 is positioned at an exterior portion of the prepuce F′ adjacent the glans G with a distal edge 216 at or just proximal to the planned incision line M. The preputial opening O may then be widened by pulling outwardly on the distal edge of the prepuce F (e.g., at two or more circumferential locations along the edge of the opening O) so that the inner member 300 can be delivered through the opening O to an interior portion of the prepuce F″. The inner member 300 is then positioned over the glans G so that the cap 308 covers and/or is adjacent to a distal portion of the glans G and the distal edges 314, 216 of the inner member 300 and band 202, respectively, are generally aligned. For example, a clinician may rotate the handle 400 (which causes rotation of the inner member 300) to achieve a desired alignment.

As shown in FIG. 10D, the band 202 of the outer member 200 can then be tightened over the inner member 300 to hold and compress the prepuce F therebetween. The clinician can use one hand to push and/or pull the arms 208, 210 in circumferentially opposing directions to increase and/or decrease the diameter D of the band 202 to a desired band diameter D. The clinician may also leverage the radially extending arms 208, 210 (e.g., with one hand) to adjust the longitudinal position of the band 202 along the shaft S. Once the desired diameter D and/or longitudinal position is reached, the engaging members 228 of the first arm 208 can engage the engaging members 228 of the second arm and lock (e.g., temporarily) the arms 208, 210 relative to one another, thereby fixing the diameter D. Should the clinician subsequently desire a different positioning of the band 202, the clinician may release the fastening portions 220, 222 by engaging (e.g., pushing) the lip 230 protruding from the first arm 208 (e.g., with one hand) to unlock the arms 208, 210 and reposition the band 202. Once repositioned, the clinician can follow the method described above to re-lock the arms. In some embodiments, the clinician may choose to remove the outer member 200 from the shaft completely before repositioning and/or re-locking. For example, the band diameter D may be widened to a point where the band 202 can be removed in a direction perpendicular to the shaft S.

As shown in the top view of FIG. 10E, the prepuce F can then be removed by cutting the prepuce F along the cutting guide (e.g., along the distal edges 216, 314 of the band 202 and inner member 300) with one or more cutting devices. In some embodiments, the flange 218 of the band 202 can include a permanent and/or actuatable sharp edge that can cut the preputial tissue. Because of the compression applied to the prepuce by the inner and outer members 200, 300, the preputial edges are held in close apposition which facilitates an accurate, straight incision as well as hemostasis. Next, one or more fastening devices can be placed at various locations and/or openings on the fastening guide (e.g., at aligned gaps 224, 304). Fastening devices can include sutures, staples, surgical glue and/or other suitable fasteners. In some embodiments, four sutures may be placed at the 3, 6, 9 and 12 o'clock positions around the edges of the prepuce F (e.g., “quadrant” sutures) using the fastening guide (e.g., at aligned gaps 224, 304 shown in dashed circles in FIG. 10E). Because of the clean incision facilitated by the cutting guide and the accuracy provided by the fastening guide, in some embodiments only simple sutures need be used. For example, the fastening guide assures accurate placement of the initial four “quadrant” sutures that can be used to guide placement of any other sutures that may prove necessary or desirable. In contrast, conventional surgical devices employ more complicated horizontal and vertical mattress sutures that are difficult to carry out by inexperienced clinicians. The circumcision assembly 100 can then be removed from the patient (e.g., either perpendicular to the shaft S or distally over the distal end of the shaft S) and, in some instances, additional fastening devices can be placed if necessary.

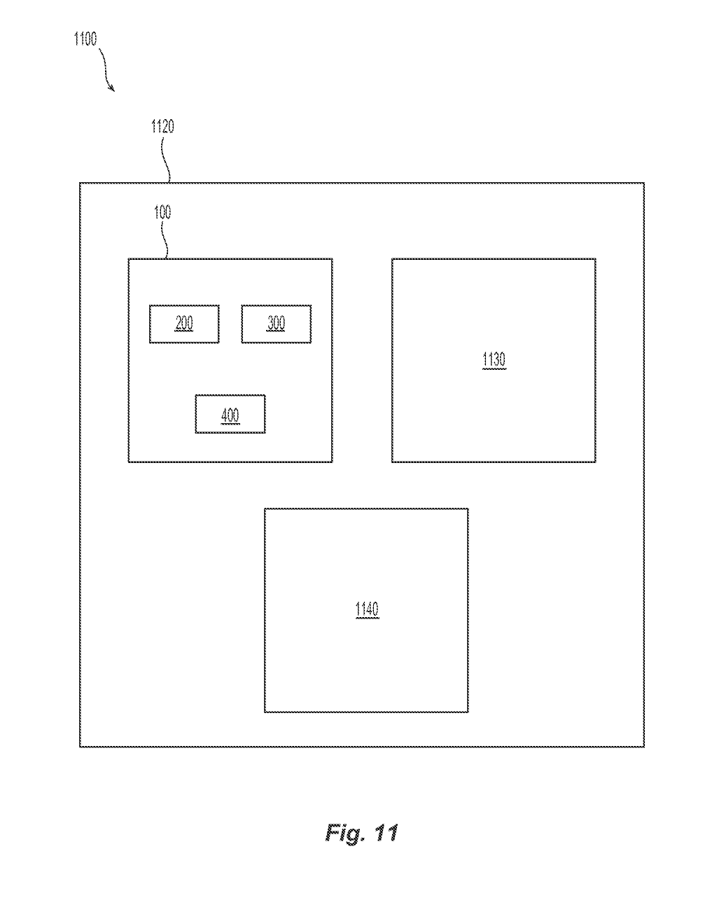

FIG. 11 is a schematic view of a kit 1100 including the circumcision assembly 100 (or another suitable circumcision assembly configured in accordance with an embodiment of the present technology) and packaging 1120. In some embodiments, the kit 1100 can further include two or more inner members (not shown) having different diameters to accommodate varying anatomies between different patients. Additionally, the kit 1100 may comprise instructions 1130 for use of the circumcision assembly 100. In some embodiments, the kit 1100 may also comprise one or more of the following: a cutting device, a fastening device, a gripping device, one or more anesthetics, a pain medication, a bandage, a medical tape, a sizing device, a marker, or an antiseptic. In other embodiments, the kit 1100 may have a different arrangement and/or include different features.

### IV. EXAMPLES

1. A circumcision device, comprising:
an outer member configured to be positioned along an exterior portion of a prepuce adjacent a glans of a penis of a human patient, the outer member including an open-ended band moveable between an unlocked position and a plurality of locked positions, wherein the band has circumferentially overlapping first and second ends in each of the locked positions, and wherein the band defines a closed space having a different fixed diameter in each of the locked positions; and
a cylindrical inner member configured to be positioned within the band of the outer member and adjacent an inner portion of the prepuce,
wherein, when the open-ended band is in one of the locked positions, the outer member and the inner member are configured to compress preputial tissue therebetween.

2. The device of example 1 wherein the open-ended band further comprises:
a first arm extending radially outwardly from the band at or near the first end of the band;
a second arm extending radially outwardly from the band at or near the second end of the band; and
wherein the first arm is configured to engage the second arm to lock the band in one of the plurality of locked positions.

3. The device of example 1 or example 2 wherein:
the band comprises a first outer region and a first inner region having a flange extending around at least a portion of a circumference of the band; and
the inner member has a second inner region and a second outer region having a groove extending around at least a portion of the circumference of the inner member;
wherein, when the outer and inner members are mated, the flange of the band is configured to engage the groove of the inner member with the preputial tissue therebetween.

4. The device of any one of examples 1-3 wherein the inner member has a cap portion configured to be positioned at or distal to a distal portion of a glans of the penis.

5. The device of any one of examples 1-3 wherein the band further comprises a plurality of castellations extending longitudinally from a distal portion of the band.

6. The device of example 5 wherein the inner member has a plurality of castellations defining a plurality of gaps therebetween, and wherein at least one of the plurality of gaps is configured to be aligned with at least one of the plurality of gaps extending longitudinally from the band.

7. The device of any one of examples 1-6 wherein the first arm has a first fastening portion and the second arm has a second fastening portion configured to engage the first fastening portion to lock the band in one of the plurality of locked positions.

8. The device of any one of examples 1-7 wherein movement of the first end of the band in one of a clockwise or a counterclockwise direction and movement of the second end of the band in the other of the clockwise or a counterclockwise directions decreases a diameter of the band.

9. The device of any one of examples 1-8 wherein the inner member is a first inner member, and wherein the device further comprises a second inner member, wherein:
the first inner member has a first diameter;
the second inner member has a second diameter less than the first diameter; and
the band of the outer member further comprises a sizing guide including—
   a first zone corresponding to the first inner member; and
   a second zone corresponding to the second inner member.

10. The device of any one of examples 1-9 wherein the outer member is one of a pediatric size, an adolescent size, or an adult size.

11. The device of any one of examples 1-10 wherein the inner member has a proximal portion and a distal portion, and wherein the device further comprises a handle configured to be detachably coupled to the distal portion of the inner member.

12. The device of example 11, further comprising a connector having a first connecting end coupled to a distal region of the handle and a second connecting end coupled to the outer member.

13. The device of example 11, further comprising a connector having a first connecting end coupled to a distal region of the inner member and a second connecting end coupled to the outer member.

14. A method for circumcising a human male patient, the method comprising:
positioning an open-ended band of a circumcision assembly around at least a portion of an outer surface of a prepuce adjacent a glans of a penis of the patient;
positioning an inner member of the circumcision assembly at an interior portion of the prepuce adjacent to the band such that a distal edge of the band is generally aligned with a distal edge of the inner member;
decreasing a diameter of the band around the inner member to compress the preputial tissue therebetween;
cutting the prepuce along a distal edge of the band; and removing a cut portion of the prepuce from the penis.

15. The method of example 14, further comprising marking the exterior surface of the prepuce along the circumference of the prepuce parallel to a corona.

16. The method of example 14, further comprising removably attaching a handle to a distal portion of the inner member and adjusting the position of the inner member relative to the band via the handle.

17. The method of example 14 wherein the outer member comprises (a) a first arm extending radially outwardly from the band at or near the first end of the band, and (b) a second arm extending radially outwardly from the band at or near the second end of the band, and wherein decreasing a diameter of the band further comprises:
moving the first arm and the second arm toward each other.

18. The method of example 14 wherein decreasing the diameter of the band further includes decreasing the diameter of the band to a first diameter, and wherein the method further comprises:
temporarily locking the band at the first diameter;
adjusting the diameter to a second diameter greater than the first diameter; and
temporarily locking the band at the second diameter.

19. The method of example 14 wherein the band further comprises a plurality of first castellations extending longitudinally from a distal portion of the band and defining a plurality of first gaps therebetween, and the inner member further comprises second castellations extending longitudinally from a distal portion of the inner member and defining a plurality of second gaps therebetween, and wherein the method further comprises:
aligning at least one of the first gaps with at least one of the second gaps.

20. The method of example 19 wherein positioning an inner member further comprises fastening a first cut end of the prepuce with a second cut end of the prepuce between at least one of the aligned first and second gaps.

21. The method of example 19, further comprising—
increasing the diameter of the band; and
repositioning the band relative to the outer surface of the prepuce before cutting the prepuce.

22. A kit, comprising:
a circumcision assembly including—
   a first member having—
      an annular portion moveable between an unlocked position and a plurality of locked positions, the annular portion having—
         a first end;
         a second end overlapping the first end when the annular portion is in each of the locked positions;
      a first arm extending radially outwardly from the annular portion at or near the first end of the first member; and
      a second arm extending radially outwardly from the annular portion at or near the second end of the first member,
      wherein the first arm is configured engage the second arm to lock the first member in one of a plurality of locked positions;
   a second member configured to be positioned within the annular portion of the first member and adjacent an inner portion of the prepuce, the second member having a planar cap portion configured to be positioned at or distal to a distal portion of a glans of a penis; and
packaging containing the circumcision assembly.

23. The kit of example 22, further comprising a handle configured to be detachably coupled to a distal portion of the second member.

24. The kit of example 22 or example 23 wherein the kit comprises two or more second members having different inner diameters.

25. The kit of any one of examples 22-24, further comprising instructions in the packaging for performing a circumcision using the circumcision assembly.

26. The kit of any one of example 22-25, further comprising one or more of the following: a cutting device, a fastening device, a gripping device, one or more anesthetics, a pain medication, a bandage, a medical tape, a sizing device, a marker, or an antiseptic.

27. A circumcision device, comprising:
an outer member configured to be positioned along an exterior portion of a prepuce adjacent a glans of a penis of a human patient, the outer member including—
   an open-ended band moveable between an unlocked position and a plurality of locked positions, wherein the band has circumferentially overlapping first and second ends in each of the locked positions;
   a first arm extending radially outwardly from the band at or near the first end of the band;
   a second arm extending radially outwardly from the band at or near the second end of the band; and
   wherein the first arm is configured engage the second arm to lock the band in one of a plurality of locked positions, and wherein the band defines a closed space having a different fixed diameter in each of the locked positions;
a cylindrical inner member configured to be positioned within the band of the outer member and adjacent an inner portion of the prepuce,
wherein, when the open-ended band is in one of the locked positions, the outer member and the inner member are configured to compress preputial tissue therebetween.

### V. CONCLUSION

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A circumcision device, comprising:
an outer member configured to be positioned along an exterior portion of a prepuce adjacent a glans of a penis of a human patient, the outer member including an open-ended band moveable between an unlocked position and a plurality of locked positions, wherein the band has circumferentially overlapping first and second ends in each of the locked positions, and wherein the band defines a closed space having a different fixed diameter in each of the locked positions; and
a cylindrical inner member configured to be positioned within the band of the outer member and adjacent an inner portion of the prepuce,
wherein, when the open-ended band is in one of the locked positions, the outer member and the inner member are configured to compress preputial tissue therebetween.

2. The device of claim 1, wherein the open-ended band further comprises:
a first arm extending radially outwardly from the band at or near the first end of the band;
a second arm extending radially outwardly from the band at or near the second end of the band; and
wherein the first arm is configured to engage the second arm to lock the band in one of the plurality of locked positions.

3. The device of claim 2, wherein:
the band comprises a first outer region and a first inner region having a flange extending around at least a portion of a circumference of the band; and
the inner member has a second inner region and a second outer region having a groove extending around at least a portion of the circumference of the inner member;
wherein, when the outer and inner members are mated, the flange of the band is configured to engage the groove of the inner member with the preputial tissue therebetween.

4. The device of claim 2, wherein the inner member has a cap portion configured to be positioned at or distal to a distal portion of a glans of the penis.

5. The device of claim 2, wherein the band further comprises a plurality of castellations extending longitudinally from a distal portion of the band.

6. The device of claim 5, wherein the inner member has a plurality of castellations defining a plurality of gaps therebetween, and wherein at least one of the plurality of gaps is configured to be aligned with at least one of the plurality of gaps extending longitudinally from the band.

7. The device of claim 2, wherein the first arm has a first fastening portion and the second arm has a second fastening portion configured to engage the first fastening portion to lock the band in one of the plurality of locked positions.

8. The device of claim 2, wherein movement of the first end of the band in one of a clockwise or a counterclockwise direction and movement of the second end of the band in the other of the clockwise or a counterclockwise directions decreases a diameter of the band.

9. The device of claim 2, wherein the inner member is a first inner member, and wherein the device further comprises a second inner member, wherein:
the first inner member has a first diameter;
the second inner member has a second diameter less than the first diameter; and
the band of the outer member further comprises a sizing guide including—
a first zone corresponding to the first inner member; and
a second zone corresponding to the second inner member.

10. The device of claim 2, wherein the outer member is one of a pediatric size, an adolescent size, or an adult size.

11. The device of claim 2, wherein the inner member has a proximal portion and a distal portion, and wherein the device further comprises a handle configured to be detachably coupled to the distal portion of the inner member.

12. The device of claim 11, further comprising a connector having a first connecting end coupled to a distal region of the handle and a second connecting end coupled to the outer member.

13. The device of claim 11, further comprising a connector having a first connecting end coupled to a distal region of the inner member and a second connecting end coupled to the outer member.

14. A method for circumcising a human male patient, the method comprising:
positioning an open-ended band of a circumcision assembly around at least a portion of an outer surface of a prepuce adjacent a glans of a penis of the patient;
positioning an inner member of the circumcision assembly at an interior portion of the prepuce adjacent to the band such that a distal edge of the band is generally aligned with a distal edge of the inner member;
decreasing a diameter of the band around the inner member to compress the preputial tissue therebetween;
cutting the prepuce along a distal edge of the band; and
removing a cut portion of the prepuce from the penis.

15. The method of claim 14, further comprising marking the exterior surface of the prepuce along the circumference of the prepuce parallel to a corona.

16. The method of claim 14, further comprising removably attaching a handle to a distal portion of the inner member and adjusting the position of the inner member relative to the band via the handle.

17. The method of claim 14, wherein the outer member comprises (a) a first arm extending radially outwardly from the band at or near the first end of the band, and (b) a second arm extending radially outwardly from the band at or near the second end of the band, and wherein decreasing a diameter of the band further comprises:
moving the first arm and the second arm toward each other.

18. The method of claim 14, wherein decreasing the diameter of the band further includes decreasing the diameter of the band to a first diameter, and wherein the method further comprises:
temporarily locking the band at the first diameter;
adjusting the diameter to a second diameter greater than the first diameter; and
temporarily locking the band at the second diameter.

19. The method of claim 14, wherein the band further comprises a plurality of first castellations extending longitudinally from a distal portion of the band and defining a plurality of first gaps therebetween, and the inner member further comprises second castellations extending longitudinally from a distal portion of the inner member and defining a plurality of second gaps therebetween, and wherein the method further comprises:
aligning at least one of the first gaps with at least one of the second gaps.

20. The method of claim 19, wherein positioning an inner member further comprises fastening a first cut end of the prepuce with a second cut end of the prepuce between at least one of the aligned first and second gaps.

21. The method of claim 19, further comprising:
increasing the diameter of the band; and
repositioning the band relative to the outer surface of the prepuce before cutting the prepuce.

22. A kit, comprising:
a circumcision assembly including—
a first member having—
an annular portion moveable between an unlocked position and a plurality of locked positions, the annular portion having—
a first end;
a second end overlapping the first end when the annular portion is in each of the locked positions;
a first arm extending radially outwardly from the annular portion at or near the first end of the first member; and
a second arm extending radially outwardly from the annular portion at or near the second end of the first member,
wherein the first arm is configured engage the second arm to lock the first member in one of a plurality of locked positions;
a second member configured to be positioned within the annular portion of the first member and adjacent an inner portion of the prepuce, the second member having a planar cap portion configured to be positioned at or distal to a distal portion of a glans of a penis; and
packaging containing the circumcision assembly.

23. The kit of claim 22, further comprising a handle configured to be detachably coupled to a distal portion of the second member.

24. The kit of claim 22, wherein the kit comprises two or more second members having different inner diameters.

25. The kit of claim 22, further comprising instructions in the packaging for performing a circumcision using the circumcision assembly.

26. The kit of claim 22, further comprising one or more of the following: a cutting device, a fastening device, a gripping device, one or more anesthetics, a pain medication, a bandage, a medical tape, a sizing device, a marker, or an antiseptic.

27. A circumcision device, comprising:
an outer member configured to be positioned along an exterior portion of a prepuce adjacent a glans of a penis of a human patient, the outer member including—
an open-ended band moveable between an unlocked position and a plurality of locked positions, wherein the band has circumferentially overlapping first and second ends in each of the locked positions;
a first arm extending radially outwardly from the band at or near the first end of the band;
a second arm extending radially outwardly from the band at or near the second end of the band; and
wherein the first arm is configured engage the second arm to lock the band in one of a plurality of locked positions, and wherein the band defines a closed space having a different fixed diameter in each of the locked positions;
a cylindrical inner member configured to be positioned within the band of the outer member and adjacent an inner portion of the prepuce,
wherein, when the open-ended band is in one of the locked positions, the outer member and the inner member are configured to compress preputial tissue therebetween.
